(19) United States
(12) Patent Application Publication
(10) Pub. No.: US 2023/0363730 A1
(43) Pub. Date: Nov. 16, 2023

(54) AIRWAY MUCUS VISUALIZATION

(71) Applicant: Vida Diagnostics, Inc., Coralville, IA (US)

(72) Inventors: Samuel W. Peterson, Topanga, CA (US); John D. Newell, JR., Port Townsend, WA (US)

(21) Appl. No.: 18/316,840

(22) Filed: May 12, 2023

Related U.S. Application Data
(60) Provisional application No. 63/341,940, filed on May 13, 2022.
(60) Provisional application No. 63/433,011, filed on Dec. 16, 2022.

(51) Int. Cl. A61B 6/00; A61B 6/03; G06T 7/70; G06T 7/00; G06T 11/00

(52) U.S. Cl. CPC A61B 6/5217; A61B 6/032; A61B 6/50; A61B 6/5223; A61B 6/463; G06T 7/70; G06T 7/0012; G06T 11/008; G06T 2207/10081; G06T 2207/30061; G06T 2200/04; G06T 2207/20092

(57) ABSTRACT

Generating a visualization of volumetric data can include receiving a set of three-dimensional CT data representative of a lung and receiving a selection of a lung segment corresponding to the subset of the CT data. Generating the visualization can include generating a multi-planar reformat (MPR) view representative of a portion of the lung intersecting a plane and generating a display image of the MPR view visually distinguishing a planar portion of the selected lung segment from a portion of the lung coplanar with the planar portion and not corresponding to the selected lung segment. Generating the visualization can include generating a topographic multi-planar reformat (tMPR) image and receiving a selection of a location within the tMPR image corresponding to a selected location in the selected lung subset. The MPR view can include the selected location in the selected lung segment.

### RELATED MATTERS

This application claims the benefit of U.S. Provisional Patent Application No. 63/341,940, filed May 13, 2022, and U.S. Provisional Patent Application No. 63/433,011, filed Dec. 16, 2022, the entire contents of each of which are incorporated herein by reference.

### BACKGROUND

Increased mucus in the large airways of the lungs is associated with several different pulmonary diseases. Mucus can build up in the airways to the point of plugging the airway lumen and impairing air flow to the more distal smaller airways of the lungs. Various therapies for clearing mucus are available and under development, making it useful to be able to quantify mucus levels, for instance, before and after treatment. Airways that are plugged by mucus can be identified on x-ray computerized tomographic (CT) scans of the thorax. In some cases, it is helpful to quantify the number of mucus plugs to determine mucus plugging severity within a lung. However, it can be time and resource intensive to count each mucus plug and determine if each mucus plug fulfills all the criteria of a true mucus plug. Additionally, it can be difficult to follow a patient's airway to determine if it is obstructed as the airway traverses throughout the lung.

### SUMMARY

Some aspects of the disclosure are directed towards systems, methods, and/or non-transitory computer readable media for generating a visualization of volumetric data, such as of three-dimensional computerized tomographic (CT) data.

In some examples, a non-transitory computer readable medium can include computer-readable instructions for causing one or more processors to perform a method for generating a visualization. In some examples, such a method includes receiving a set of three-dimensional CT data representative of a lung and receiving a selection of a lung segment that corresponds to the subset of the set of CT data.

The method can include generating a multi-planar reformat (MPR) view from the CT data. The MPR view can be a view of CT data representative of a portion of the lung intersecting a plane that extends through the lung. In some such examples, the MPR view includes (i) a planar portion of the selected lung segment, and (ii) a portion of the lung, coplanar with the planar portion, not corresponding to the selected lung segment.

The method can include generating a display image of the MPR view that visually distinguishes the planar portion of the selected lung segment from the portion of the lung coplanar with the planar portion and not corresponding to the selected lung segment.

In some examples, the display image includes a plurality of MPR views. For instance, in some examples, the display image includes a first MPR view, a second MPR view, and a third MPR view. In some such examples, each MPR view comprises a view of CT data of a portion of the lung intersecting a different plane.

In some examples, the method can include generating a topographic multi-planar reformat (tMPR) image from the set of CT data, which can include a two-dimensional representation of the selected lung segment. In some examples, the display image includes the tMPR image.

In some examples, methods can include receiving a selection of a location within the tMPR image that corresponds to a selected location in the selected lung segment. The MPR view can be a view of CT data representative of a portion of the lung intersecting a plane that extends through the lung and includes the selected location of the selected lung segment. In some such examples, the MPR view includes (i) a planar portion of the selected lung segment containing the selected location, and (ii) a portion of the lung, coplanar with the planar portion, not corresponding to the selected lung segment.

In some examples, a method includes identifying a location of a candidate artifact present within an airway within the CT data and generating a tMPR image representative of a section of the CT data including the candidate artifact a portion of the airway including the candidate artifact. In some embodiments, identifying the location of the candidate artifact can include receiving a selection of a location via a user interface or detecting, for example, a mucus plug candidate using a neural network or receiving a selection of

In some examples, such methods can include generating a MPR view of the CT data including CT data within a first plane and including a portion of the candidate artifact, wherein the first plane is defined by a shape of the candidate artifact or a shape of the airway including the candidate artifact. The method can include generating a display image simultaneously comprising the tMPR image and the MPR view. In some examples, the display image simultaneously comprises the tMPR image and a plurality of MPR views, and in some examples, a plane of one MPR view is normal to the plain of another MPR view.

### BRIEF DESCRIPTION OF THE DRAWINGS

FIG. 1A is a scan of a portion of a lung with an airway being collapsed.

FIG. 1B is a scan of a portion of the lung with a mucus plug in an airway.

FIG. 2 is a scan of the portion of the lung with the airway being open.

FIG. 3 is a scan of a portion of a lung with multiple airways ending and/or disappearing against a resolution of the scan.

FIG. 4 is a perspective view of an airway blocked by a mucus plug with generated views of the mucus plug comprising a plane that extends perpendicular to a short axis and parallel to a long axis and a secondary axis of the airway according to an aspect of the present disclosure.

FIG. 5 is a perspective view of an airway blocked by a mucus plug with generated views of the mucus plug comprising a plane that extends perpendicular to a secondary axis and parallel to a long axis and a short axis of the airway according to an aspect of the present disclosure.

FIG. 6 is a perspective view of an airway blocked by a mucus plug with generated views of the mucus plug comprising a plane that extends perpendicular to a long axis and parallel to a short axis and a secondary axis of the airway according to an aspect of the present disclosure.

FIG. 7 is a perspective view of an airway blocked by a mucus plug with generated views of the mucus plug comprising a plane that extends parallel to a long axis and a secondary axis of the mucus plug according to an aspect of the present disclosure.

FIG. 8 is a perspective view of an airway blocked by a mucus plug with generated views of the mucus plug comprising a plane that extends parallel to a long axis and a short axis of the mucus plug according to an aspect of the present disclosure.

FIG. 9 is a perspective view of an airway blocked by a mucus plug with generated views of the mucus plug comprising a plane that extends parallel to a short axis and a secondary axis of the mucus plug according to an aspect of the present disclosure.

FIG. 10 is a flowchart illustrating an example method for qualifying and/or quantifying mucus plugging of a lung using generated image data according to an aspect of the present disclosure.

FIG. 11 is an example flowchart of a method of identifying characteristics of a patient's airways according to an embodiment of the present disclosure.

FIG. 12 is another example flowchart of a method of identifying characteristics of a patient's airways according to an embodiment of the present disclosure.

FIG. 13 is an example view of a patient's lung tissue and airways comprising MPR views and a tMPR view according to an embodiment of the present disclosure.

FIG. 14 is another example view of a patient's lung tissue and airways comprising MPR views and a tMPR view according to an embodiment of the present disclosure.

### DETAILED DESCRIPTION

As described herein, a multi-step process can be used to qualify and/or quantify mucus plugging. In some examples, the process is a two-step process with a first step including identifying mucus plug candidates in the airways of a patient's lungs and optionally determining the location of the mucus plug candidates. The second step can include manual confirmation/rejection of each mucus plug candidate by using generated image data of the mucus plug candidates. Further processing can be done after the multi-step process. For instance, after confirming/rejecting each mucus plug candidate, they can be counted and used to inform treatment decisions for clearing the mucus plugs. However, in some examples, the process is a one-step process with assistance, whereby generated image data is used to aid a user in examining airways of a lung, determining if a blockage or other airway characteristic is present, and marking the blockage or other airway characteristic. The assistance can further help keep track of airways and/or lung segments that have been examined and those yet needing examination.

In some examples, the first step of identifying mucus plug candidates in the airways of a patient's lungs can be done automatically. In one such example, a neural network can be implemented with deep learning to detect mucus plug candidates. The neural network can be trained on images (e.g., CT scans) of airways with mucus plugs and airways without mucus plugs to determine if an airway terminates in a mucus plug. Further, the neural network can be trained on images in which the airway is obstructed for a reason other than a mucus plug. For instance, an airway may appear blocked by a mucus plug in one image when in actuality, the image does not resolve the airway any further (e.g., resolution limit of the CT scan). In other examples, an airway may appear blocked by a mucus plug when it is collapsed or obstructed by something other than a mucus plug. After training, the neural network can be used to identify mucus plug candidates in images (e.g., CT scans) of different patients. In some examples, the neural network can be adjusted to have a higher sensitivity, whereby it identifies more false positives, or a lower sensitivity, whereby it may miss some mucus plugs. It can be preferable to have a higher sensitivity neural network as mucus plug candidates can be reviewed by an expert to confirm that they are mucus plugs while any missed mucus plugs may affect the measurement of mucus plugging severity.

While a neural network can be used to automatically identify mucus plug candidates, other types of computerized analysis can be used to automatically identify mucus plug candidates. For instance, in some examples, forms of artificial intelligence other than neural networks are used to detect, identify, and/or confirm mucus plug candidates. Additionally or alternatively, in some examples, non-artificial intelligence computerized analysis is used to detect, identify, and/or confirm mucus plug candidates. Additionally or alternatively, in some examples, the location of a patient's previously identified mucus plugs can be used as presumptive locations of their current mucus plugs. Computerized analysis can be used with the presumptive locations to further refine locations of mucus plug candidates.

Alternatively, in some examples, one or more people can manually identify mucus plug candidates. However, instead of an expert manually identifying mucus plug candidates, non-experts can be trained to identify possible mucus plug candidates which can then be reviewed by an expert later. By having non-experts identify mucus plug candidates, the time and resources required by the expert are reduced such that the expert need only review mucus plug candidates. In some examples, non-experts can be another step in the process whereby the non-experts review mucus plug candidates identified by a neural network before providing their results to an expert for final review.

Current neural networks and non-experts may not be accurate enough to detect all mucus plugs without any false positives and as such, expert review can be necessary. However, even if a neural network or non-experts are accurate enough to detect all mucus plugs with no false positives, it can be desirable for an expert to confirm the accuracy of the findings. In some examples, during an initial detection, a neural network and/or non-expert may not be able to identify if a candidate mucus plug fits the desired criteria of a mucus plug due to limitations of the imaging (e.g., CT scan slice). For instance, the desired criteria can require an airway to be open proximal to and distal to a mucus blockage while a neural network and/or non-expert might only determine, based on imaging, that the airway is open proximal to the mucus blockage. In another example, the desired criteria can require an airway to be completely blocked by mucus to constitute a mucus blockage while a neural network and/or non-expert cannot distinguish an almost fully blocked airway from a fully blocked airway (e.g., due to limitations of imaging). In many instances, one image (e.g., CT scan slice) does not contain enough information to definitively determine that a mucus plug exists. However, in some examples, a topographic multi-planar reformat (tMPR) image or “cut surface display” may display enough information to definitively determine that a mucus plug exists. The tMPR image, though, might not be able to catch all mucus plugs in the lungs. tMPR imaging of lungs, or cut-surface display of tubular structures, is discussed further in U.S. Pat. No. 11,176,666, filed Nov. 9, 2018, which is assigned to the Applicant of the instant application, the content of which is hereby incorporated by reference in its entirety.

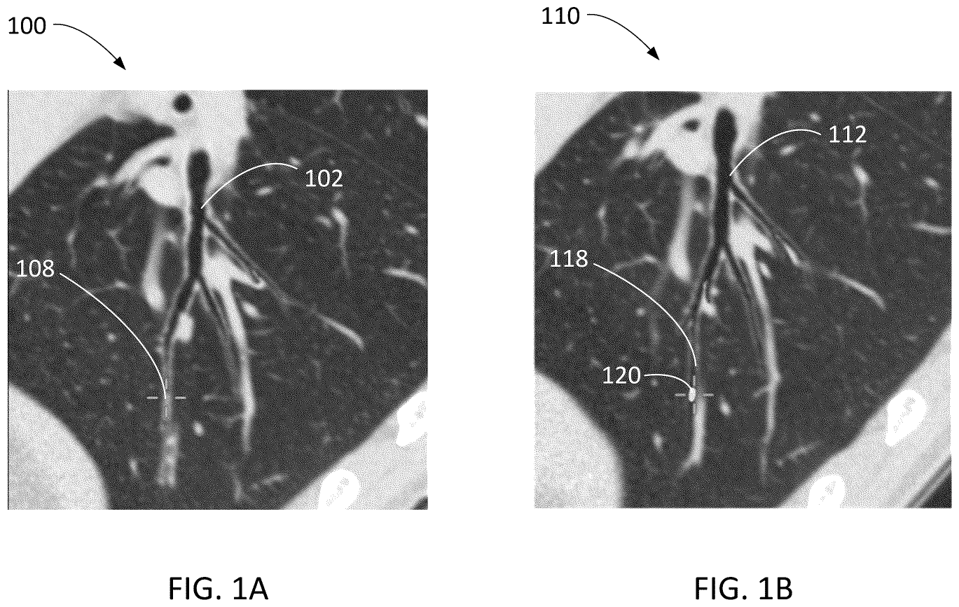

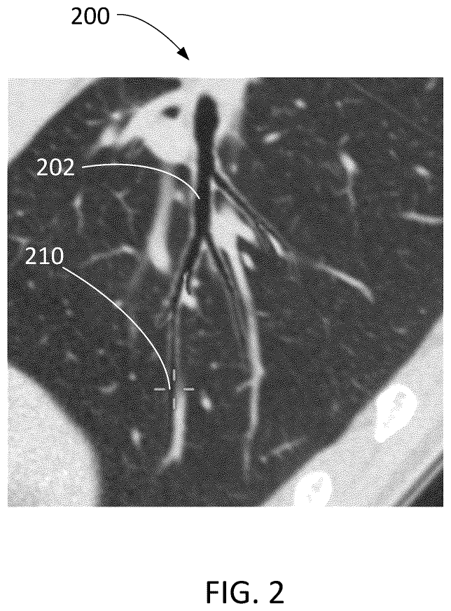

tMPR images of airways are provided in FIG. 1A-FIG. 3 and illustrate how mucus plugs appear in the tMPR imaging compared to images without mucus plugs. For instance, FIG. 1B shows a scan of a portion of lung having an airway with a sub tree of the airway being blocked by a mucus plug. In comparison, FIG. 1A and FIG. 2 show scans of a similar portion of lung with the sub tree of the airway clear from the mucus plug. However, such other obstructions may be present, for example, due to the airway being collapsed.

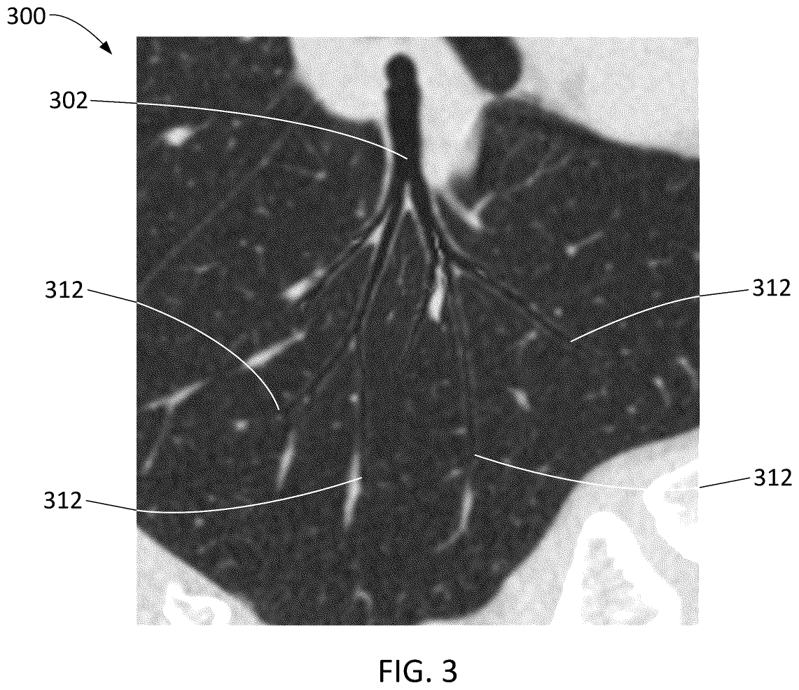

FIG. 1A is a scan 100 of a portion of a lung with a sub tree 108 of an airway 102 being collapsed. FIG. 1B is a scan 110 of a similar portion of a lung with a sub tree 118 of an airway 112 being blocked with a mucus plug 120. FIG. 2 is a scan 200 of a similar portion of lung as showing FIGS. 1A and 1B with the sub tree 210 of the airway being open. As one of ordinary skill will appreciate, a collapsed airway may be detected by a neural network or non-expert as being a false positive mucus plug candidate. Turning to FIG. 3, FIG. 3 is a scan 300 of a portion of a lung with multiple sub trees 312 of an airway 302 ending and/or disappearing against a resolution of the scan 300. It can be difficult for a neural network or non-expert to distinguish mucus plugs from an airway ending and/or disappearing against the resolution of the scan which can lead to false positives or missed plugs.

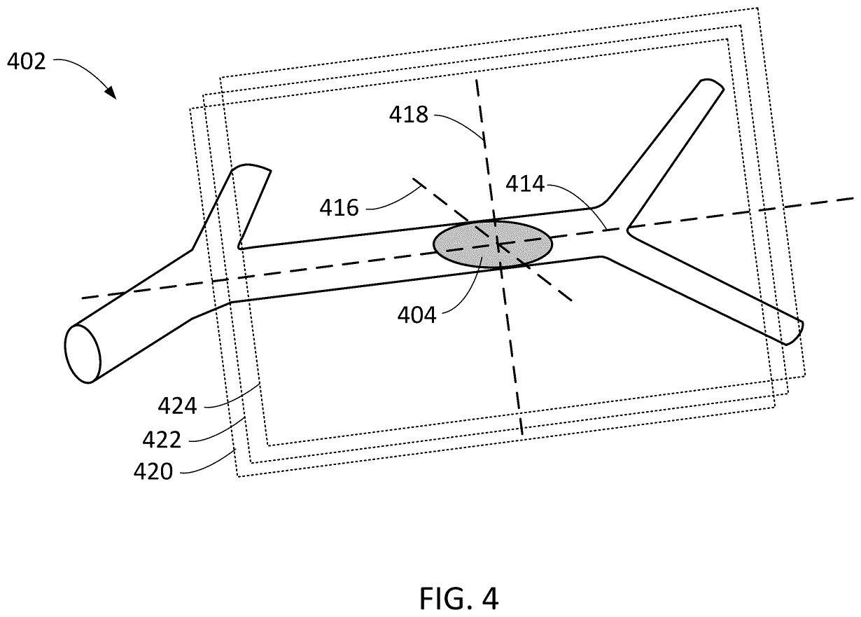

After mucus plug candidates have been identified by a neural network, a non-expert, or a combination of both, an expert can review the mucus plug candidates to determine which candidates are actual mucus plugs and which candidates are something else. To aid in the review, an optimized viewing scheme comprising image data of the area of the mucus plug candidate, including the mucus plug candidate itself can be generated. The viewing scheme can comprise a series of images such as multi-planar reformat (MPR) views, curved MPR views, and/or tMPR views. In some examples, the viewing scheme includes generating four sets of views with three of the four sets being MPR or curved MPR views with the fourth set being tMPR views. Other combinations of views are contemplated. An example of an optimized viewing scheme is illustrated in FIG. 4-FIG. 6 which depict three sets of MPR views taken along different axes.

Starting with FIG. 4, FIG. 4 is a perspective view of an airway 402 blocked by a mucus plug 404 with generated views 420, 422, 424 of the mucus plug along a long axis 414 of the airway 402 according to an aspect of the present disclosure. The airway 402 can have three defined axes including a long axis 414, a short axis 416, and a secondary axis 418 which is defined as being perpendicular to both the long axis 414 and the short axis 416 (e.g., cross-product of the long axis 414 and the short axis 416). In some examples, the axes can be defined arbitrarily, however, in some examples, the axes correlate with the orientation and variation in the shape of the airway at the location of the plug. For instance, in FIG. 4-FIG. 6, the long axis is defined along the direction in which the airway exhibits the widest variation, the short axis is defined along the direction in which the airway exhibits the least variation, and the secondary axis is defined by the cross-product of the long axis and the short axis. Further, in FIG. 4-FIG. 6, a center of the axes (e.g., origin) is defined by the centroid of the mucus plug. In some examples, though, a center of the axes is defined by the center of the interface between air and mucus within the plug. Defining the axes in the illustrated way can keep the definition of axes consistent for mucus plugs in different airways which can be useful for generating sets of images of the mucus plugs.

Continuing with FIG. 4, the set of generated views 420, 422, 424 are sectional views which encompass the mucus plug 404 and surrounding area of the mucus plug 404. The generated views 420, 422, 424 can be centered on the mucus plug 404 but need not be. Each generated view comprises a plane that extends parallel to the long axis 414 and the secondary axis 418 and which is normal to the short axis 416 (e.g., has a different short axis coordinate). For example, generated view 420 is at a different short axis coordinate than generated view 422 or 424. Each generated view of the generated views 420, 422, 424 thus runs through a different portion of the mucus plug 404 and provides a different sectional view of the mucus plug 404. An expert can use such generated views 420, 422, 424 to help them determine if a mucus plug candidate is actually a mucus plug or if it is something else. For instance, in some examples, the generated views 420, 422, 424 can be displayed on a display and allow an expert to traverse back and forth through the generated views 420, 422, 424 to determine if the mucus plug 404 fulfills the criteria for a mucus plug (e.g., has an open airway distal to the mucus plug). Having multiple sectional views of the mucus plug and being able to go back and forth through the views can provide valuable information to an expert to aid in their confirmation/rejection of a mucus plug candidate. While only three generated views are used in the illustrated example, any number of views can be used, though the number of views can be limited by the scanning technology. For instance, CT scans can have a fixed resolution which does not enable more than a certain number of sectional views. Further, the generated views can be larger or smaller than those illustrated in FIG. 4. Larger generated views can enable an expert to see further out from a mucus plug candidate which can be beneficial if a mucus plug obstructs multiple airways (e.g., a bifurcation of an airway); determining if the airways open up after the mucus plug can be one criterion for confirming a mucus plug candidate is a mucus plug.

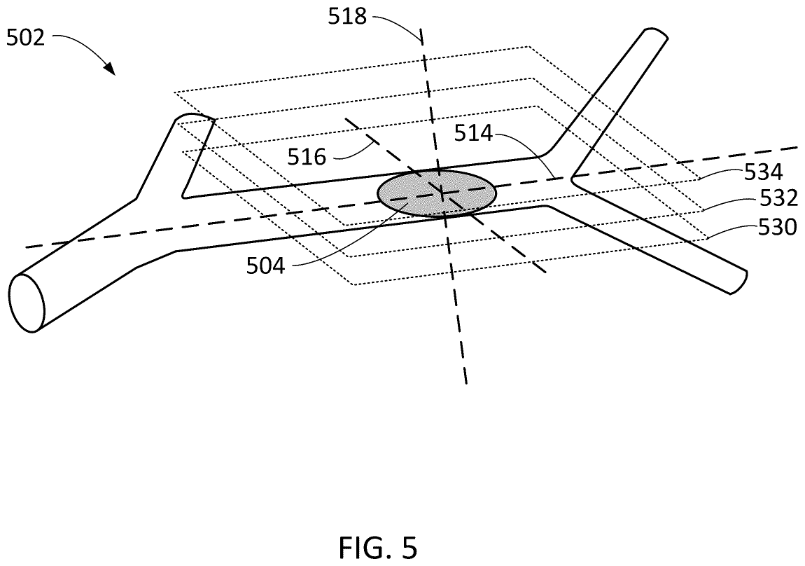

Moving to FIG. 5, FIG. 5 is a perspective view of an airway 502 blocked by a mucus plug 504 with generated views 530, 532, 534 of the mucus plug along a short axis 516 of the airway 502 according to an aspect of the present disclosure. In similarity with FIG. 4, the airway 502 can have three defined axes including a long axis 514, a short axis 516, and a secondary axis 518 being perpendicular to both the long and short axes 514, 516. The set of generated views 530, 532, 534 are sectional views which encompass the mucus plug 504 and surrounding area of the mucus plug 504. The set of generated views 530, 532, 534 can be centered on the mucus plug 504 but need not be. Each generated view comprises a plane that extends parallel to the long axis 514 and the short axis 516 and which is normal to the secondary axis 518 (e.g., has a different secondary axis coordinate). For example, generated view 530 is at a different secondary axis coordinate than generated view 532 or 534. Each generated view of the generated views 530, 532, 534 thus runs through a different portion of the mucus plug 504 and provides a different sectional view of the mucus plug 504. An expert can use such generated views 530, 532, 534 to help them determine if a mucus plug candidate is actually a mucus plug or if it is something else. For instance, in some examples, the generated views 530, 532, 534 can be displayed on a display and allow an expert to traverse back and forth through the generated views 530, 532, 534 to determine if the mucus plug 504 fulfills the criteria for a mucus plug (e.g., has an open airway distal to the mucus plug). Having multiple sectional views of the mucus plug and being able to go back and forth through the views can provide valuable information to an expert to aid in their confirmation/rejection of a mucus plug candidate. While only three generated views are used in the illustrated example, any number of views can be used, though the number of views can be limited by the scanning technology. For instance, CT scans can have a fixed resolution which does not enable more than a certain number of sectional views. Further, the generated views can be larger or smaller than those illustrated in FIG. 5. Larger generated views can enable an expert to see further out from a mucus plug candidate which can be beneficial if a mucus plug obstructs multiple airways (e.g., a bifurcation of an airway); determining if the airways open up after the mucus plug can be one criterion for confirming a mucus plug candidate is a mucus plug. As one of ordinary skill will appreciate, the generated views 530, 532, 534 of FIG. 5 can be used in addition to, or in lieu of the generated views 420, 422, 424 of FIG. 4 to aid an expert in confirming the presence of a mucus plug in an airway.

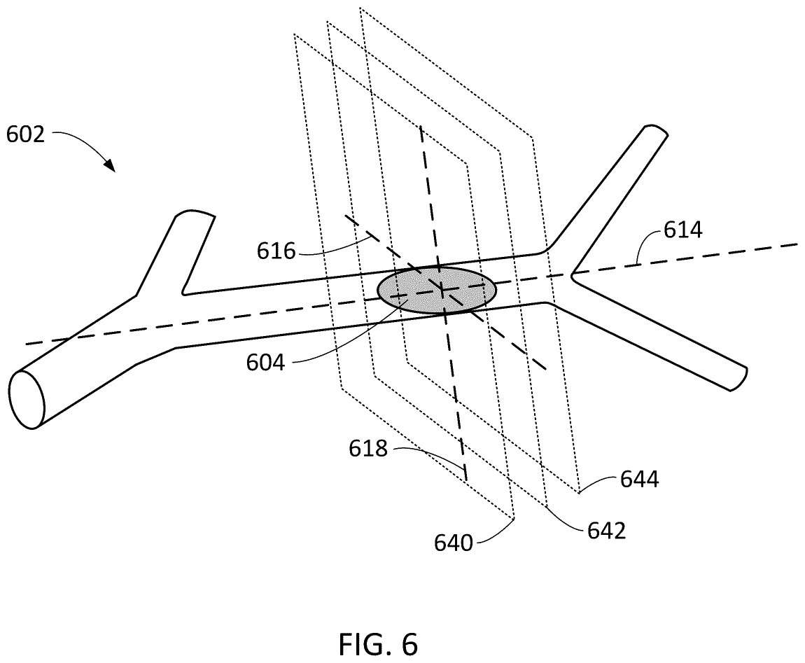

Moving to FIG. 6, FIG. 6 is a perspective view of an airway 602 blocked by a mucus plug 604 with generated views 640, 642, 644 of the mucus plug along a secondary axis 618 of the airway 602 according to an aspect of the present disclosure. In similarity with FIG. 4 and FIG. 5, the airway 602 can have three defined axes including a long axis 614, a short axis 616, and the secondary axis 618 which is perpendicular to both the long and short axes 614, 616. The set of generated views 640, 642, 644 are sectional views which encompass the mucus plug 604 and surrounding area of the mucus plug 604. The set of generated views 640, 642, 644 can be centered on the mucus plug 604 but need not be. Each generated view comprises a plane that extends parallel to the short axis and the secondary axis and which is normal to the long axis (e.g., has a different long axis coordinate). For example, generated view 640 is at a different long axis coordinate than generated view 642 or 644. Each generated view of the generated views 640, 642, 644 thus runs through a different portion of the mucus plug 604 and provides a different sectional view of the mucus plug 604. An expert can use such generated views 640, 642, 644 to help them determine if a mucus plug candidate is actually a mucus plug or if it is something else. For instance, in some examples, the generated views 640, 642, 644 can be displayed on a display and allow an expert to traverse back and forth through the generated views 640, 642, 644 to determine if the mucus plug 604 fulfills the criteria for a mucus plug (e.g., has an open airway distal to the mucus plug). Having multiple sectional views of the mucus plug and being able to go back and forth through the views can provide valuable information to an expert to aid in their confirmation/rejection of a mucus plug candidate. While only three generated views are used in the illustrated example, any number of views can be used, though the number of views can be limited by the scanning technology. For instance, CT scans can have a fixed resolution which does not enable more than a certain number of sectional views. Further, the generated views can be larger or smaller than those illustrated in FIG. 6. Larger generated views can enable an expert to see further out from a mucus plug candidate which can be beneficial if a mucus plug obstructs multiple airways (e.g., a bifurcation of an airway); determining if the airways open up after the mucus plug can be one criterion for confirming a mucus plug candidate is a mucus plug. As one of ordinary skill will appreciate, the generated views 640, 642, 644 of FIG. 6 can be used in addition to, or in lieu of the generated views 420, 422, 424 of FIG. 4 and the generated views 530, 532, 534 of FIG. 5 to aid an expert in confirming the presence of a mucus plug in an airway.

As discussed above, FIG. 1B shows a tMPR scan of an airway with a mucus plug (120), while FIGS. 1A and 2 show a scan of an airway without a mucus plug. The tMPR views that capture the mucus plug can be used in addition to, or in lieu of the sets of generated MPR views of FIG. 4-FIG. 6 to aid an expert in confirming if a mucus plug candidate is actually a mucus plug. The tMPR views can provide valuable information about the airway proximal to the mucus plug as the tMPR view is “warped” to represent the three-dimensional airway onto a two-dimensional surface (e.g., a flat image). In some examples, a tMPR view may extend distal to the mucus plug and provide information on the airway directly following the mucus plug. In some such examples, the generated tMPR view alone can enable an expert to confirm if a mucus plug candidate is a mucus plug.

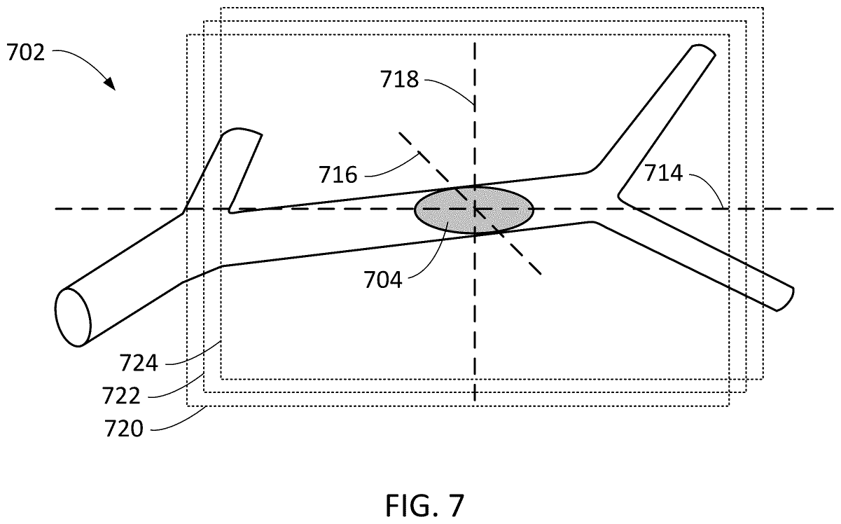

Moving to FIG. 7-FIG. 9, FIG. 7-FIG. 9 are perspective views of airways blocked with mucus plugs with views of the mucus plug generated along various axes defined relative to the mucus plug, not relative to the airway as is FIG. 4-FIG. 6. Generating views relative to the mucus plug can be advantageous in some situations when compared to generating views relative to airway.

Starting with FIG. 7, FIG. 7 is a perspective view of an airway 702 blocked by a mucus plug 704 with generated views 720, 722, 724 of the mucus plug along a long axis 714 of the mucus plug 704 according to an aspect of the present disclosure. The mucus plug 704 can have three defined axes include a long axis 714, a short axis 716, and a secondary axis 718 which is defined as being perpendicular to both the long axis 714 and the short axis 716 (e.g., cross-product of the long axis 714 and the short axis 716). In some examples, the axes can be defined arbitrarily, however, in some examples, the axes correlate with the variation in the shape of the mucus plug (or mucus plug candidate). For instance, in FIG. 7-FIG. 9, the long axis is defined along the direction in which the mucus plug exhibits the widest variation, the short axis is defined along the direction in which the mucus plug exhibits the least variation, and the secondary axis is defined by the cross-product of the long axis and the short axis. Further, in FIG. 7-FIG. 9, a center of the axes (e.g., origin) is defined by the centroid of the mucus plug. In some examples, though, a center of the axes is defined by the center of the interface between air and mucus within the plug. Defining the axes in the illustrated way can keep the definition of axes consistent for mucus plugs in different airways which can be useful for generating sets of images of the mucus plugs.

Continuing with FIG. 7, the set of generated views 720, 722, 724 are sectional views which encompass the mucus plug 704 and surrounding area of the mucus plug 704. The set of generated views 720, 722, 724 can be centered on the mucus plug 704 but need not be. Each generated view comprises a plane that extends parallel to the long axis 714 and the secondary axis 718 and which is normal to the short axis 716 (e.g., has a different short axis coordinate). For example, generated view 720 is at a different short axis coordinate than generated view 722 or 724. Each generated view of the generated views 720, 722, 724 thus runs through a different portion of the mucus plug 704 and provides a different sectional view of the mucus plug 704. An expert can use such generated views 720, 722, 724 to help them determine if a mucus plug candidate is actually a mucus plug or if it is something else. For instance, in some examples, the generated views 720,722, 724 can be displayed on a display and allow an expert to traverse back and forth through the generated views 720, 722, 724 to determine if the mucus plug 704 fulfills the criteria for a mucus plug (e.g., has an open airway distal to the mucus plug). Having multiple sectional views of the mucus plug and being able to go back and forth through the views can provide valuable information to an expert to aid in their confirmation/rejection of a mucus plug candidate. While only three generated views are used in the illustrated example, any number of views can be used, though the number of views can be limited by the scanning technology. For instance, CT scans can have a fixed resolution which does not enable more than a certain number of sectional views. Further, the generated views can be larger or smaller than those illustrated in FIG. 7. Larger generated views can enable an expert to see further out from a mucus plug candidate which can be beneficial if a mucus plug obstructs multiple airways (e.g., a bifurcation of an airway); determining if the airways open up distal to the mucus plug can be one criterion for confirming a mucus plug candidate is a mucus plug.

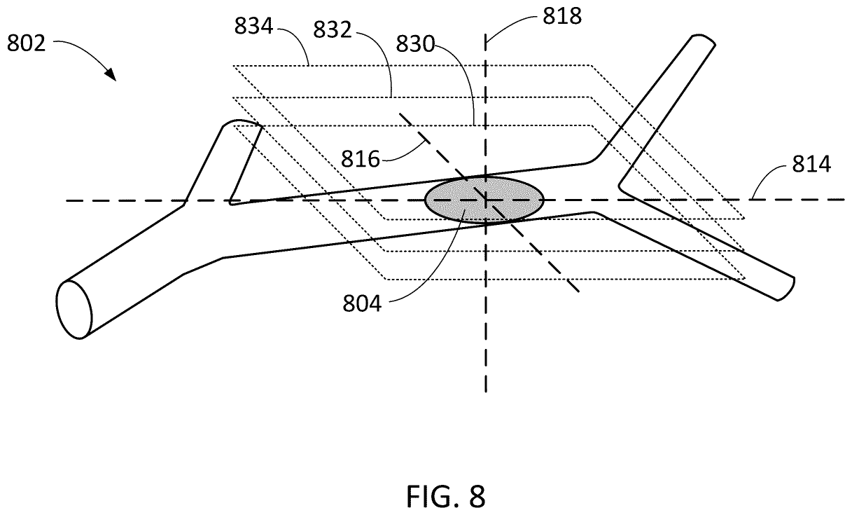

Moving to FIG. 8, FIG. 8 is a perspective view of an airway 802 blocked by a mucus plug 804 with generated views 830, 832, 834 of the mucus plug along a short axis 816 of the mucus plug 804 according to an aspect of the present disclosure. In similarity with FIG. 7, the mucus plug 804 can have three defined axes including a long axis 814, a short axis 816, and a secondary axis 818 being perpendicular to both the long and short axes 814, 816. The set of generated views 830, 832, 834 are sectional views which encompass the mucus plug 804 and surrounding area of the mucus plug 804. The set of generated views 830, 832, 834 can be centered on the mucus plug 804 but need not be. Each generated view comprises a plane that extends parallel to the long axis 814 and the short axis 816 and which is normal to the secondary axis 818 (e.g., has a different secondary axis coordinate). For example, generated view 830 is at a different secondary axis coordinate than generated view 832 or 834. Each generated view of the generated views 830, 832, 834 thus runs through a different portion of the mucus plug 804 and provides a different sectional view of the mucus plug 804. An expert can use such generated views 830, 832, 834 to help them determine if a mucus plug candidate is actually a mucus plug or if it is something else. For instance, in some examples, the generated views 830, 832, 834 can be displayed on a display and allow an expert to traverse back and forth through the generated views 830, 832, 834 to determine if the mucus plug 804 fulfills the criteria for a mucus plug (e.g., has an open airway distal to the mucus plug). Having multiple sectional views of the mucus plug and being able to go back and forth through the views can provide valuable information to an expert to aid in their confirmation/rejection of a mucus plug candidate. While only three generated views are used in the illustrated example, any number of views can be used, though the number of views can be limited by the scanning technology. For instance, CT scans can have a fixed resolution which does not enable more than a certain number of sectional views. Further, the generated views can be larger or smaller than those illustrated in FIG. 8. Larger generated views can enable an expert to see further out from a mucus plug candidate which can be beneficial if a mucus plug obstructs multiple airways (e.g., a bifurcation of an airway); determining if the airways open up after the mucus plug can be one criterion for confirming a mucus plug candidate is a mucus plug. As one of ordinary skill will appreciate, the generated views 830, 832, 834 of FIG. 8 can be used in addition to, or in lieu of the generated views 720, 722, 724 of FIG. 7 to aid an expert in confirming the presence of a mucus plug in an airway.

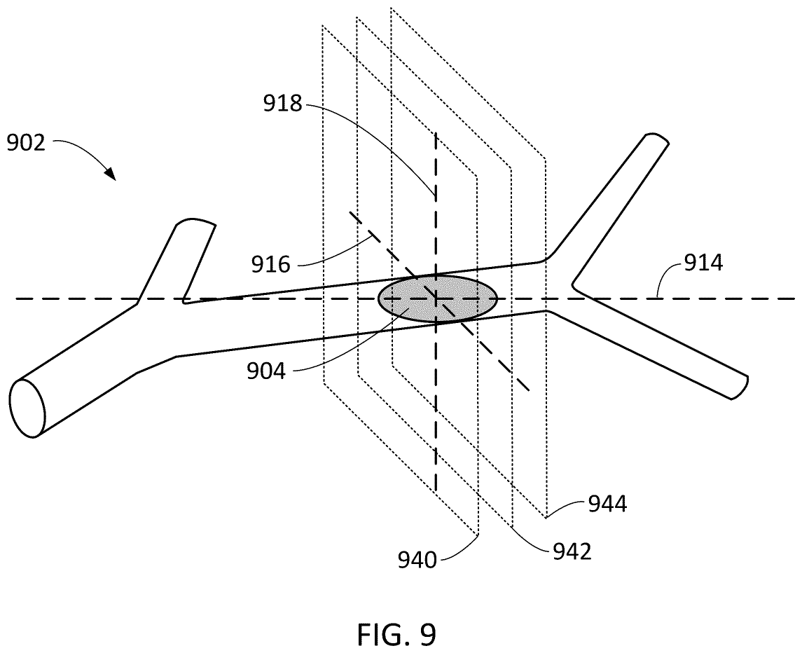

Moving to FIG. 9, FIG. 9 is a perspective view of an airway 902 blocked by a mucus plug 904 with generated views 940, 942, 944 of the mucus plug along a secondary axis 918 of the mucus plug 904 according to an aspect of the present disclosure. In similarity with FIG. 7 and FIG. 8, the mucus plug 904 can have three defined axes including a long axis 914, a short axis 916, and the secondary axis 918 which is perpendicular to both the long and short axes 914, 916. The set of generated views 940, 942, 944 are sectional views which encompass the mucus plug 904 and surrounding area of the mucus plug 904. The set of generated views 940, 942, 944 can be centered on the mucus plug 904 but need not be. Each generated view comprises a plane that extends parallel to the short axis and the secondary axis and which is normal to the long axis (e.g., has a different long axis coordinate). For example, generated view 940 is at a different long axis coordinate than generated view 942 or 944. Each generated view of the generated views 940, 942, 944 thus runs through a different portion of the mucus plug 904 and provides a different sectional view of the mucus plug 904. An expert can use such generated views 940, 942, 944 to help them determine if a mucus plug candidate is actually a mucus plug or if it is something else. For instance, in some examples, the generated views 940, 942, 944 can be displayed on a display and allow an expert to traverse back and forth through the generated views 940, 942, 944 to determine if the mucus plug 904 fulfills the criteria for a mucus plug (e.g., has an open airway distal to the mucus plug). Having multiple sectional views of the mucus plug and being able to go back and forth through the views can provide valuable information to an expert to aid in their confirmation/rejection of a mucus plug candidate. While only three generated views are used in the illustrated example, any number of views can be used, though the number of views can be limited by the scanning technology. For instance, CT scans can have a fixed resolution which does not enable more than a certain number of sectional views. Further, the generated views can be larger or smaller than those illustrated in FIG. 9. Larger generated views can enable an expert to see further out from a mucus plug candidate which can be beneficial if a mucus plug obstructs multiple airways (e.g., a bifurcation of an airway); determining if the airways open up after the mucus plug can be one criterion for confirming a mucus plug candidate is a mucus plug. As one of ordinary skill will appreciate, the generated views 940, 942, 944 of FIG. 9 can be used in addition to, or in lieu of the generated views 420, 422, 424 of FIG. 7 and the generated views 830, 832, 834 of FIG. 8 to aid an expert in confirming the presence of a mucus plug in an airway. For instance, a comprehensive view of a mucus plug candidate is created by using three different sets of generated MPR views taken from three different directions (e.g., along three axes that are perpendicular to each other) each of which contains multiple images of the mucus plug at different depths, heights, or widths. This comprehensive view can be used by an expert to confirm or reject a mucus plug candidate with greater accuracy than other imaging or methods.

In some examples, a processor is configured to generate the sets of MPR views and tMPR views in the form of image data. In some such examples, a display can receive the generated image data and display the MPR views and tMPR views for a user to see and/or traverse through. In some examples, a non-transitory computer-readable medium can have instructions stored thereon that, when executed by a processor, cause the processor to generate, for an individual mucus plug candidate, one or more tMPR views and three sets of MPR views, along three axes as illustrated in the examples of FIG. 4-FIG. 6. The views can be generated in the form of image data which can be output. In some examples, the image data is output to a display, however, in some examples, the image data is output to a processor that can further manipulate the image data (e.g., using algorithms). One such example can be a processor configured to generate and output the image data of the mucus plug candidate to another processor configured to eliminate some mucus plug candidates from the image data.

As described elsewhere herein with respect to FIG. 4-FIG. 9, in some examples, an expert can traverse generated MPR views which encompass a section or “slice” of a mucus plug candidate and surrounding airway. While traversing the generated views can be valuable for confirming a mucus plug, other imaging techniques, such as volume rendering techniques, may remove the need to traverse through multiple sections or may provide better visualization for an expert to confirm a mucus plug candidate. For instance, in some examples, maximum intensity projection (MIP) imaging or minimum intensity projection (MinIP) imaging can be used. In some such examples, instead of generating a thin section of the mucus plug (e.g., as illustrated in FIG. 4-FIG. 9), a thicker “slab” section is generated which comprises image data from multiple, parallel thin sections. For example, each thin section of a mucus plug candidate and surrounding airway can have a thickness of 0.6 mm while a thicker slab section can have a thickness of 5.0 mm. Thus, the thicker slab section contains between 9-10 thin sections of image data. To display the image data of every thin section, the thicker slab section can use MIP imaging or MinIP imaging. In the case of MIP imaging, all the CT values from a single point location on the thin sections are aggregated with the largest CT number being displayed as part of the MIP image. This process is repeated for all points within the thin slice sections until a full MIP image is generated. In contrast, MinIP imaging aggregates all the CT values from a single point location on the thin sections with the smallest CT number being displayed as part of the MinIP image. Again, this process is repeated for all points within the thin slice sections until a full MinIP image is generated.

In general, the MIP or MinIP image is generated and/or displayed as a two-dimensional image and thus collapses one of the three spatial dimensions (e.g., axes) associated with a three-dimensional object (e.g., mucus plug candidate). In doing so, image data from each thin section can be displayed simultaneously on the MIP or MinIP image. To avoid generating images with excessive overlap of structures, or to include more structures in generated images, the thickness of the slab section can be decreased or increased respectively. For example, increasing the thickness may provide more context to a mucus plug candidate including the ability to follow an airway through the body, while decreasing the thickness may remove excess airways which are confusing or not useful to confirm/reject a mucus plug candidate. Further, in some examples, the thicker slab of a MIP or MinIP image can be translated along the axis collapsed by the MIP or MinIP. In one such example, a MIP or MinIP image runs along a plane defined by a long axis (e.g., 414) and a secondary axis (e.g., 416) and collapses the short axis (e.g., 418). The thicker slab of the MIP or MinIP image can then be translated along the short axis (e.g., 418) to display a continuous set of MIP or MinIP images of the area. In similarity with the thin section viewing described elsewhere herein, the MIP or MinIP image data can be sent to a display for viewing wherein an expert or other person can traverse the MIP or MinIP images to confirm a mucus plug. Using MIP or MinIP can be particularly useful in helping an expert or other person visualize a three dimensional mucus plug candidate to confirm if it is indeed a mucus plug.

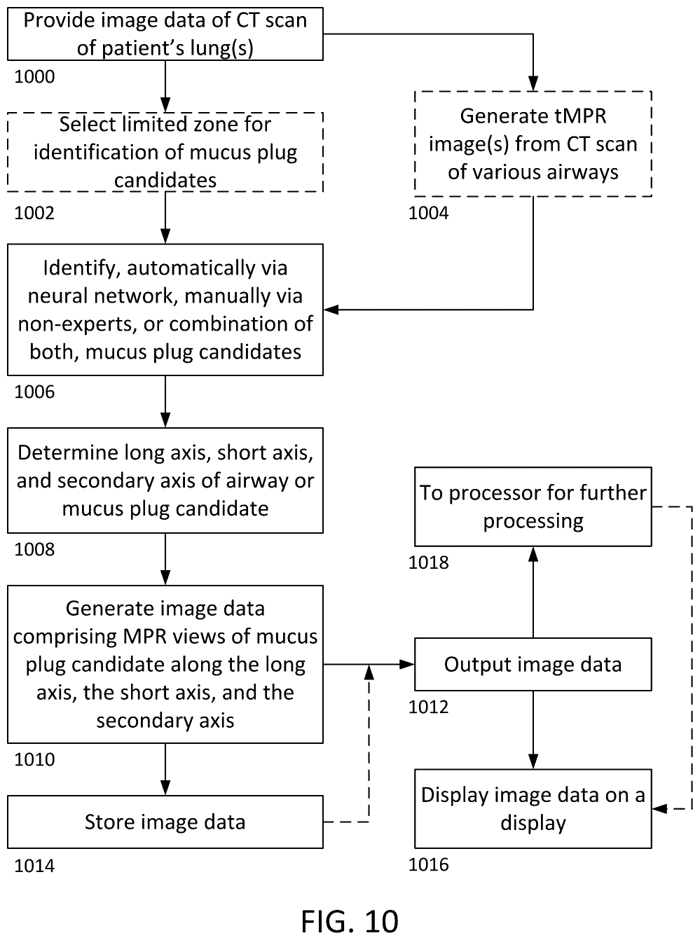

Moving to FIG. 10, FIG. 10 is a flowchart of a process for generating image data of mucus plug candidates. Starting at step 1000, image data generated from a CT scan of a patient's lung or lungs can be provided. Continuing with optional step 1002, a non-expert, an algorithm, a neural network, or similar entity can select a zone of a patient's lung(s) to limit the extent of mucus plug identification. In some examples, a user can select a sublobar region or regions of a lung to limit identification of mucus plug candidates or alternatively, a user can select a region or regions of the lung to exclude from identification of mucus plug candidates. In some such examples, various regions of a lung can be displayed for a user (e.g., a non-expert) for them to select which region to limit mucus plug identification. Visualization and analysis of such sublobar regions of a lung are further described in U.S. patent application Ser. No. 13/439,387 filed Apr. 4, 2012, now abandoned, which is assigned to the Applicant of the instant application, the content of which is hereby incorporated by reference in its entirety. Additionally or alternatively, plugs may be enumerated or counted separately in the various zones of the lung and an aggregate score may be computed based on the presence and/or number of plugs within each individual zone. Limiting identification of mucus plug candidates to specified regions of a lung can be useful in reducing time and resource cost when only a portion of lung needs mucus plugs to be identified.

Continuing with another optional step 1004, tMPR image(s) of various airways can be generated from the CT scan image data. As discussed elsewhere herein, the tMPR images can be used for context in confirming mucus plugs. Further in step 1006, mucus plug candidates are identified automatically via a neural network, manually via non-experts, or some combination of automatically and manually. When combined with optional step 1004, step 1006 includes identifying mucus plug candidates only in a limited zone or zones of a patient's lung. Next, in step 1008, the long axis, short axis, and secondary axis of an airway having a mucus plug candidate, or of the mucus plug candidate itself, are determined. This can be done by, for example, determining the amount of variation along an airway. Continuing on, in step 1010, image data comprising MPR views of a mucus plug candidate is generated. The MPR views can include sets of MPR views from the long axis, the short axis, and the secondary axis which can be determined in step 1008. After generation of the image data comprising MPR views and optional tMPR views, the image data can be output as in step 1012 or stored as in 1014. Image data stored in step 1014 can later be output as in step 1012 as indicated by the dashed arrow. The image data generated in step 1010 can be output to a variety of places, but in step 1016, the generated image data is displayed on a display while in step 1018, the image data is output to a processor for further processing of the image data. Optionally, after further processing of the image data, the processed image data can be displayed on a display as in step 1016. While the steps of the flowchart in FIG. 10 are illustrated as happening in order, a person having ordinary skill will appreciate that the steps may be executed in nearly any order and some steps may happen simultaneously. Additionally, FIG. 10 may exclude steps that are contemplated (e.g., marking mucus plug candidates to easily locate them) and/or combine steps which may be separable.

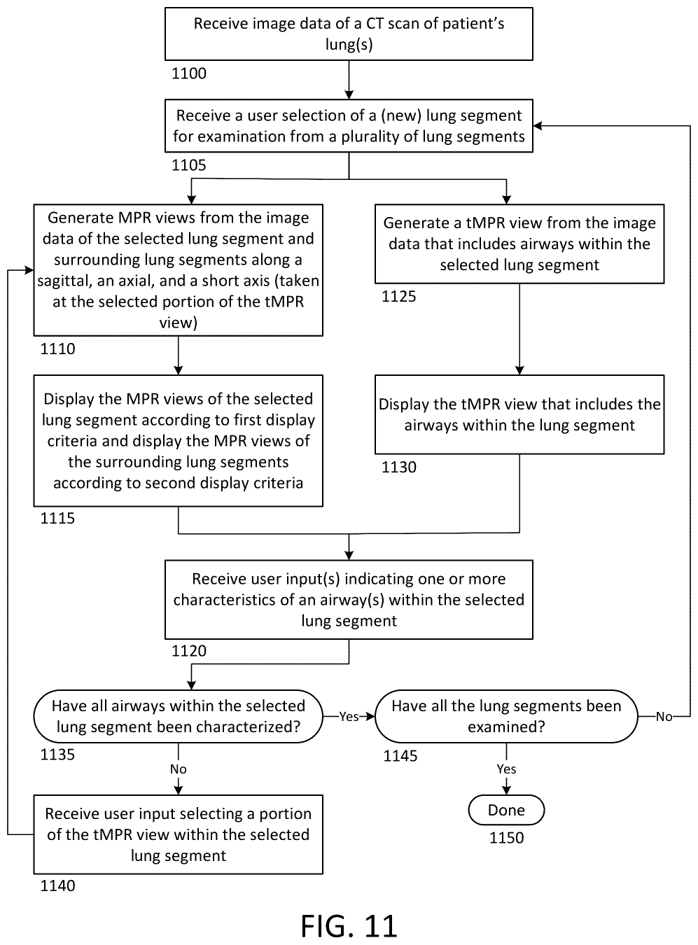

While a multi-stage workflow is described elsewhere herein, in some examples, a single stage workflow can be used to identify blockages (e.g., mucus plugs) within a patient's lungs. In some such examples, instead of first identifying mucus plug candidates and subsequently having the mucus plug candidates reviewed/confirmed by a radiologist, a radiologist can immediately identify and confirm mucus plugs in a patient's lungs. This can be advantageous as it can reduce the overall time to confirm the number of mucus plugs within a patient's lungs. For instance, an example workflow is illustrated in the flowchart of FIG. 11.

FIG. 11 is an example flowchart of a method of identifying characteristics of a patient's airways according to an embodiment of the present disclosure. In the example, a system performs the steps of the method. Starting at step 1100, a system can receive image data of a CT scan of a patient's lungs. The CT image data can include, for example, multiple CT slices of a patient's chest.

Next, in step 1105, the system can receive a user selection of a lung segment from a plurality of lung segments. A lung segment, as used herein, is generally defined as a region of the lung corresponding to a group of one or more bronchi and descendent branches thereof. In some examples, the plurality of lung segments comprise between 18 to 20 lung segments depending on the anatomy of the patient and/or the airway grouping used. The system can receive the user selection via a user interface such as a drop down menu displayed on a display, for example.

Once selected, the system can generate MPR views from the image data of the selected lung segment and surrounding lung segments along various axes as in step 1110. In the embodiment of FIG. 11, the system can generate MPR views of the selected lung segment and surrounding lung segments along a sagittal axis, an axial axis (e.g., transverse axis), and a short axis. The sagittal axis and the axial axis are the commonly understood axes of the human body while the short axis can be determined separately. In some examples, the short axis can be the coronal axis. However, in some examples, such as the embodiment illustrated in FIG. 11, the short axis is defined as being perpendicular to the largest airway within the selected segment. Other alternatives for defining the short axis are contemplated including, but not limited to: defining the short axis as being perpendicular to an average direction of the airways within a selected lung segment, defining the short axis as being perpendicular to a majority of the airways within the selected segment, and defining the short axis as being perpendicular to an airway closest to a selected portion of a tMPR view.

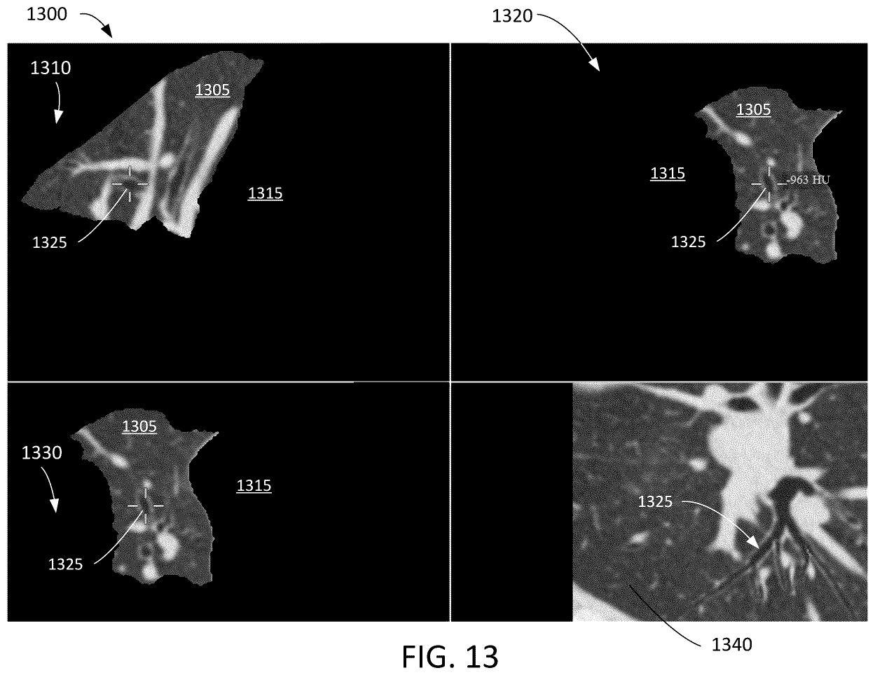

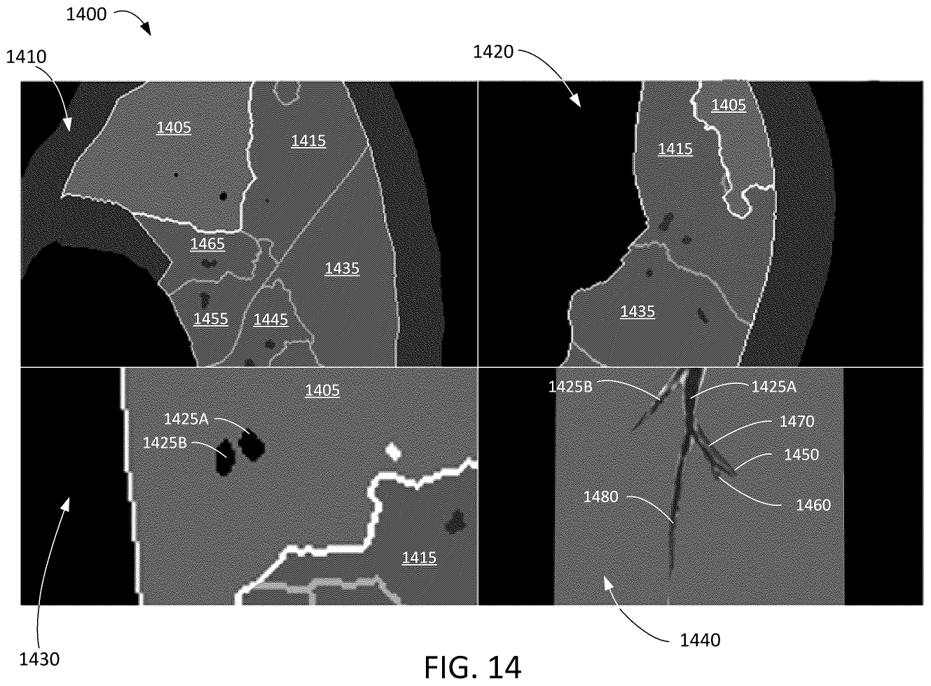

After the MPR views are generated, the system can display the MPR views (e.g., on a display) as in step 1115. In the example of FIG. 11, all three MPR views can be displayed simultaneously. FIG. 13 and FIG. 14 are examples that include three MPR views being displayed simultaneously. Additionally, the MPR views of the selected lung segment are displayed according to first display criteria while the MPR views of the surrounding lung segments (e.g., all segments that are not the selected lung segment) are displayed according to second display criteria. The first display criteria are generally different from the second display criteria. The first and second display criteria can be chosen from a variety of display criteria including, but not limited to: color, transparency, focus/blur, obfuscation, and brightness. The first and second display criteria can each comprise multiple criteria (e.g., color and transparency) or a single criterion (obfuscation). Having different display criteria can help a user more easily follow an airway present in the selected lung segment and distinguish the selected lung segment from surrounding lung segments. For instance, traditional MPR views do not distinguish different lung segments from each other. Accordingly, a radiologist, when examining an airway within a specific lung segment for mucus plugs, may find it difficult and/or time consuming to distinguish the airway within the specific lung segment from other airways in other lung segments in the MPR views. Without distinguishing display criteria, the radiologist may need to review various MPR views multiple times to ensure that the airway they are examining for a mucus plug is the airway in the specific lung segment and not another airway in another lung segment.

In one example of step 1115, displaying the MPR views of the selected lung segment according to first display criteria can comprise simply displaying the MPR views of the selected lung segment on a display. In the same example, displaying the MPR views of the surrounding lung segments according to second display criteria can comprise obfuscating (e.g., not displaying) those surrounding segments. FIG. 13 illustrates such an example. In another example, displaying the MPR views of the selected lung segment according to first display criteria can comprise displaying the MPR views with a silver-tinted transparency. In such an example, displaying the MPR views of the surrounding lung segments according to second display criteria can comprise displaying the MPR views with a violet-tinted transparency. FIG. 14 illustrates such an example.

In some examples, displaying the MPR views of the selected lung segment according to first display criteria can be toggled on/off by a user (e.g., user input to the system). Similarly, in some examples, displaying the MPR views of the surrounding lung segments according to second display criteria can be toggled on/off by the user. When toggled off, the MPR views will illustrate image data unaltered by display criteria (e.g., raw CT imagery). Toggling the first display criteria on/off and toggling the second display criteria on/off can be done independently of each other or simultaneously. For instance, in some embodiments, the first display criteria comprising simply displaying the MPR views of the selected lung segment can be left toggled on while the second display criteria comprising obfuscating the surrounding lung segments can be toggled off and back on by a user input to the system. Toggling the display criteria on/off can enable a user to view the unaltered MPR views which can provide important context that may be less visible to the user when the MPR views are displayed according to specific display criteria.

Continuing with step 1120, the system can receive one or more user inputs indicating one or more characteristics of one or more airways within the selected lung segment. In the example of FIG. 11, the one or more characteristics of the one or more airways within the selected lung segment can include: a presence and/or location of a mucus plug, a presence and/or location of bronchiectasis, a presence and/or location of an occlusion (e.g., not definitively a mucus plug), whether an airway is unscoreable, and whether an airway has no finding (e.g., none of the listed characteristics). In a specific example, a user can provide an input to the system that indicates a selected airway within the selected lung segment has a mucus plug along with a location of the mucus plug within the selected airway. In some examples, a user can provide further inputs to the system such as written comments that are associated with an input indicating a characteristic of an airway. A person having ordinary skill in the art will appreciate that other characteristics are contemplated, and that this disclosure is not limited to the example characteristics listed above.

The one or more user inputs can be provided to the system in a variety of ways including via human interface devices (HIDs) such as a computer mouse. Further, the one or more user inputs received by the system can comprise a user marking a location within a selected airway that is within the selected lung segment. The location within the selected airway can be defined in three-dimensional coordinates such that the exact location of the marking is identifiable by the system. For example, a user can mark a location within one of the MPR views that indicates a mucus plug is present in that location. The system can then determine/receive the three-dimensional coordinate of the mark made by the user in addition to the information that the mark is the location of a mucus plug.

The system can also incorporate memory that is configured to store the one or more user inputs. For example, the system can store a mark made by the user including the three-dimensional coordinate of the mark. Further, the system can store the type of mark along with any extra comments associated with the mark. In one such example, a user can mark a location of a blockage within an airway and add a comment associated with the mark that the blockage needs further examination to determine if it is a mucus plug. In this example, the system can store the mark type (i.e., a blockage), the mark location (i.e., three-dimensional coordinates of the mark), and the comment associated with the mark. By storing the one or more user inputs, the stored one or more user inputs can be viewed/retrieved later, ensuring that a user does not need to analyze/mark the MPR views another time.

The step 1120 can be repeated many times for each airway within the selected lung segment. For example, the system can receive a user input indicating a mucus plug in one airway within the selected lung segment and a user input indicating no finding (e.g., no blockage) in a different airway within the selected lung segment.

Moving to steps 1125 and 1130, the system can generate a tMPR view from the image data that includes airways within the selected lung segment and can subsequently display the tMPR view. As described elsewhere herein, the tMPR view can illustrate an airway as it splits from a main portion (e.g., trachea) to smaller portions (e.g., segmental bronchi). In some examples, displaying the tMPR view can be done at the same time as displaying the MPR views. For instance, as illustrated in the examples of FIG. 13 and FIG. 14, the three MPR views taken along a sagittal, an axial, and a short axis are displayed simultaneously with the tMPR view. Displaying the tMPR view at the same time as the MPR views can aid a user in identifying airways such as a selected airway within a selected lung segment. The tMPR view can also be used to navigate airways as is further described with respect to step 1140. From step 1130, the method continues with step 1120 as the steps of generating and displaying the tMPR view can occur at or around the same time as the steps of generating and displaying the MPR views.

After step 1120, the system moves to step 1135. Step 1135 recites “have all airways within the selected lung segment been characterized?” Step 1135 is a decision step which does not need to be actively performed by the system. If the answer to the decision step is “no,” the method continues with step 1140. However, if the answer to the decision step is “yes,” the method continues with step 1145.

Continuing with step 1140, the system can receive a user input selecting a portion of the tMPR view within the selected lung segment. The system can then return to step 1110 to generate new MPR views of the selected lung segment and surrounding lung segments along a sagittal, an axial, and a short axis, taken at the selected portion of the tMPR view. In this way, a user can select a desired portion of the tMPR view and have corresponding MPR views of the desired portion generated and displayed. This can be beneficial as a user can use the tMPR view, which generally outlines and follows airways, to follow an airway as it continues through the lung. As a user follows the airway, the user can view different angles of the airway by interacting with a portion of the tMPR view to generate corresponding MPR views along the sagittal, the axial, and the short axes. Using the tMPR view and the MPR views, the user can indicate (e.g., mark) one or more characteristics of the airway such as indicating a mucus plug is present in an airway. The tMPR views and the MPR views can enable a user to easily find and mark locations of mucus plugs.

As described elsewhere herein, the short axis can be defined as being perpendicular to an airway (e.g., largest airway) within the selected lung segment. Accordingly, the MPR view taken along a short axis can change as a user selects a different portion of the tMPR view. For instance, in some examples, the MPR view taken along the short axis can update such that the MPR view is perpendicular to the nearest airway selected in the portion of the tMPR view. In some such examples, a user can select a portion of the tMPR view that includes an airway within the selected lung segment and the MPR view will update to be perpendicular to that airway.

The method following steps 1110 through 1140 can be repeated until all the airways within the selected lung segment have been characterized. Once all the airways within the selected lung segment have been characterized, the method continues with the decision step 1145.

Step 1145 is a decision step which does not need to be actively performed by the system. Step 1145 recites “have all the lung segments been examined?” If the answer to the decision step is “yes,” then the method ends with step 1150. However, if the answer to the decision step is “no,” the method returns to step 1105 whereby the system receives a user selection of a new lung segment for examination from a plurality of lung segments. The method again continues with steps 1110 through 1145 until all the lung segments have been examined. In some examples, the system of FIG. 11 can perform the method for between 18 and 20 different lung segments. However, in some examples, the method can be limited to a portion of lung segments and need not be performed for all lung segments. For example, the method can be limited to segments within one or more lobes of the lungs or limited to segments of a left lung or right lung (e.g., 8 to 10 lung segments.)

While steps in the example of FIG. 11 are illustrated and described as being sequential, one of ordinary skill in the art will appreciate that not all steps need to be performed sequentially as some steps can occur before others and some steps can occur substantially simultaneously. For example, the system that performs the method of FIG. 11 can generate tMPR views of all lung segments before a user selects a lung segment. Additionally or alternatively, not all the steps of the method of FIG. 11 are required to be performed. For example, a user may not need to indicate a characteristic of an airway within the selected lung segment as in step 1120 if, for instance, no airways are blocked. Additionally or alternatively, some steps or series of steps may be repeated before moving to the next step. For example, the system can receive user input selecting a portion of the tMPR view within the selected lung segment, generate MPR views taken at the selected portion, and display the MPR views multiple times before the system receives user input(s) indicating one or more characteristics of an airway or airways within the selected lung segment.

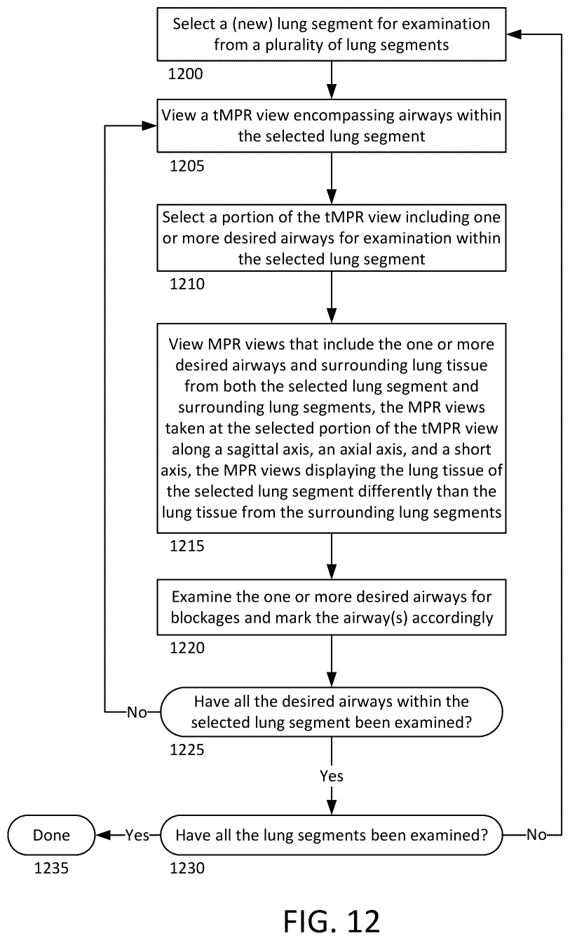

Moving to FIG. 12, FIG. 12 is another example flowchart of a method of identifying characteristics of a patient's airways according to an embodiment of the present disclosure. In contrast to the method of FIG. 11 in which a system performs the steps, the method of FIG. 12 is taken from a perspective of a user performing the steps. In some examples, the user is a trained radiologist. However, in some examples, the user can be a non-medical professional that is trained to identify characteristics of airways within lungs of a patient.

Starting with step 1200, a user can select a lung segment for examination from a plurality of lung segments. Next, in step 1205, the user can view a tMPR view that encompasses airways within the selected lung segment. As described elsewhere herein, the tMPR view can be a cut plane through CT imagery that generally follows one or more airways as they extend through the lung. The user can then select a portion of the tMPR view that includes one or more desired airways for examination and that are within the selected lung segment as in step 1210. For example, as described with respect to FIG. 11, the user can use a computer mouse to engage a portion of the tMPR view that includes an airway the user wants to examine.

Continuing with step 1215, the user can view MPR views that include the one or more desired airways and surrounding lung tissue from both the selected lung segment and surrounding lung segments. The MPR views can be taken at the selected portion of the tMPR view along a sagittal axis, an axial axis, and a short axis. Accordingly, the user can view three different MPR views that are taken at the selected portion of the tMPR view simultaneously. Further, in some examples, the tMPR view is also displayed along with the MPR views. As such, the user can view the three MPR views taken along different axes and the tMPR view simultaneously.

Further in step 1215, the MPR views can display the lung tissue and airways of the selected lung segment differently than the lung tissue and airways from the surrounding lung segments. As discussed elsewhere herein, the MPR views of the selected lung segment can be displayed according to first display criteria and the MPR views of the surrounding lung segments can be displayed according to second display criteria. The first display criteria and the second display criteria being different can aid a user in following airways within a selected lung segment without getting confused with airways present in surrounding lung segments.

Continuing with step 1220, the user can examine the one or more desired airways for blockages (e.g., mucus plugs) and mark the airway(s) accordingly. For instance, in some examples, a user can mark an airway with one of five options including: a mucus plug, bronchiectasis, occluded airway, unscoreable, or no finding. The user can mark the airway(s) with an indicator (e.g., a crosshair) that is easily distinguishable from the CT imagery displayed in the MPR/tMPR views. In some examples, the user can add additional details (e.g., written comments) that are associated with the mark. The additional details can be stored by the system as described elsewhere herein.

Continuing with step 1225, the user can decide if all the desired airways within the selected lung segment have been examined and/or marked. If the user determines that not all the desired airways within the selected lung segment have been examined/and or marked, the method returns to step 1205 and continues through steps 1205 to 1225 until all the desired airways have been examined and/or marked. Once the user determines that all the desired airways within the selected lung segment have been examined, the user can determine if all the lung segments have been examined as in step 1230. If not all the lung segments have been examined, the method can return to step 1200 whereby a user selects a new lung segment for examination. If all the lung segments have been examined, the method finishes with step 1235.

While steps in the example of FIG. 12 are illustrated and described as being sequential, one of ordinary skill in the art will appreciate that not all steps need to be performed sequentially as some steps can occur before others and some steps can occur substantially simultaneously. For example, the user may view a tMPR view encompassing airways within the selected lung segment simultaneously as viewing MPR views of airways within the selected lung segment. Additionally or alternatively, not all the steps of the method of FIG. 11 are required to be performed. For example, a user might not need to select a new lung segment for examination as the system can automatically select a new lung segment after all airways within a selected lung segment are examined and marked.

Moving to FIG. 13, FIG. 13 is an example overall view 1300 of a patient's lung tissue and airways comprising MPR views 1310, 1320, 1330, and a tMPR view 1340 according to an embodiment of the present disclosure. As illustrated, the top left MPR view 1310 is taken along a sagittal axis, the top right MPR view 1320 is taken along an axial axis, and the bottom left MPR view 1330 is taken along a short axis. Accordingly, the top left MPR view 1310 is also referred to as a “sagittal MPR view”, the top right MPR view 1320 is also referred to as an “axial MPR view”, and the bottom left MPR view 1330 is also referred to as a “short axis MPR view.”

The bottom right view is a tMPR view 1340. The MPR views 1310, 1320, 1330 and the tMPR view 1340 include CT imagery of lung tissue. The example overall view 1300 includes all the MPR views 1310, 1320, 1330 and the tMPR view 1340. The example overall view 1300 can be presented to a user on one or more displays such that the user can see all three MPR views and the tMPR view simultaneously. As described elsewhere herein, being able to view the three MPR views and the tMPR view at the same time or on the same display can aid a user in examining the airways of a patient's lungs.

Focusing on the MPR views, the sagittal MPR view 1310 includes a selected lung segment 1305 that includes an airway 1325. The sagittal MPR view 1310 also includes non-selected lung segment(s)/surrounding lung segment(s). The selected lung segment 1305 is displayed according to first display criteria while the non-selected lung segment(s) are displayed according to second display criteria. In the sagittal MPR view 1310, the first display criteria include simply displaying the CT image data of the selected lung segment 1305 while the second display criteria includes obfuscating the CT image data of the non-selected lung segment(s). The obfuscation includes completely blacking out the non-selected lung segment(s). In many examples, the obfuscation blocks out multiple lung segments, however, in some examples, the obfuscation blocks a single lung segment. In some examples, the obfuscation can be toggled on or off according to a user input as is described elsewhere herein.

Further in FIG. 13, the axial MPR view 1320 and the short axis MPR view 1330 both illustrate the same lung segment 1305, including the same airway 1325, as in the sagittal MPR view 1310. The airway 1325 is illustrated in the three MPR views 1310, 1320, 1330 which are taken along the three different axes. By displaying the airway 1325 along three different axes, a user can more clearly see the airway and if the airway 1325 has a blockage (e.g., a mucus plug.)

Additionally, the non-selected lung segment(s) 1315 are also obfuscated in the axial MPR view 1320 and the short axis MPR view 1330. While not shown, in some examples, one or two of the three displayed MPR views can obfuscate the non-selected lung segments(s) while the other one or two of the MPR views can display the non-selected lung segment(s). For example, a sagittal MPR view can obfuscate non-selected lung segment(s) while the axial and short axis MPR views do not obfuscate non-selected lung segment(s). Additionally or alternatively, the obfuscation of the non-selected lung segment(s) can be toggled on or off for each of the three MPR views independently from each other.

In some examples, one or more views further obfuscate CT data representing lung tissue within a predetermined margin from the chest wall, such as within 2 cm of the chest wall. Additionally or alternatively, in some examples, data proximate the chest wall is excluded from analysis, such as when identifying one or more candidate objects such as a mucus plug.

Moving to the tMPR view 1340, the tMPR view can display the same airway 1325 as is displayed in the three MPR views 1310, 1320, 1330. As described elsewhere herein, a user can interact with the tMPR view 1340 to navigate to different airways within a selected lung segment and/or different portions of the selected lung segment. The tMPR view 1340 can aid a user in navigating airways within a lung segment and can display airways that need further examination using the MPR views.

Moving to FIG. 14, FIG. 14 is another example overall view 1400 of a patient's lung tissue and airways comprising MPR views 1410, 1420, 1430 and a tMPR view 1440 according to an embodiment of the present disclosure. As illustrated, the top left MPR view 1410 is taken along a sagittal axis, the top right MPR view 1420 is taken along an axial axis, and the bottom left MPR view 1430 is taken along a short axis. Accordingly, the top left MPR view 1410 is also referred to as a “sagittal MPR view”, the top right MPR view 1420 is also referred to as an “axial MPR view”, and the bottom left MPR view 1430 is also referred to as a “short axis MPR view.”

As illustrated, FIG. 14 includes an example overall view 1400 that includes a selected lung segment 1405 and a plurality of non-selected/surrounding lung segments 1415, 1435, 1445, 1455, 1465. The selected lung segment 1405 includes airways 1425A and 1425B which are displayed in the short axis MPR view 1430 and the tMPR view 1440. Some of the non-selected/surrounding lung segments can be seen in multiple MPR views (e.g., 1415) along with some of their airways.

The example of FIG. 14 is similar to the example of FIG. 13 in that the selected lung segment 1405 is displayed according to first display criteria which include simply displaying the CT image data of the selected lung segment 1405. However, the example of FIG. 14 is different than the example of FIG. 13 in that instead of the second display criteria comprising obfuscation, thereby blacking out any non-selected lung segments, the second display criteria comprise highlighting non-selected lung segments in a color different than the selected lung segment. In FIG. 14, the color is an indigo highlight which is semi-transparent. A semi-transparent coloring of the non-selected/surrounding lung segments can be advantageous as it enables a user to view the context of the non-selected/surrounding lung segments even if the user is primarily interested in a selected lung segment.

Various non-limiting embodiments have been described and are within the scope of the overall disclosure.

1. A non-transitory computer readable medium comprising computer-readable instructions for causing one or more processors to perform a method for generating a visualization of volumetric data, the method comprising:
receiving a set of three-dimensional computerized tomographic (CT) data representative of a lung;
receiving a selection of a lung segment that corresponds to a subset of the set of CT data;
generating a multi-planar reformat (MPR) view from the CT data, the MPR view being a view of CT data representative of a portion of the lung intersecting a plane extending through the lung, such that the MPR view includes (i) a planar portion of the selected lung segment and (ii) a portion of the lung, coplanar with the planar portion, not corresponding to the selected lung segment; and
generating a display image of the MPR view, the display image visually distinguishing the planar portion of the selected lung segment from the portion of the lung coplanar with the planar portion and not corresponding to the selected lung segment.

2. The non-transitory computer readable medium of claim 1, wherein the MPR view comprises a first MPR view, the first MPR view being a view of a portion of the lung intersecting a first plane such that the first MPR view includes (i) a first planar portion of the selected lung segment containing the selected location and (ii) a portion of the lung, coplanar with the first planar portion, not corresponding to the selected lung segment, and wherein the method further comprises:
generating a second multi-planar reformat (MPR) view from the CT data, the second MPR view being a view of CT data representative of a portion of the lung intersecting a second plane extending through the lung that includes the selected location in the selected lung segment, such that the second MPR view includes (i) a second planar portion of the selected lung segment containing the selected location and (ii) a portion of the lung, coplanar with the second planar portion, not corresponding to the selected lung segment; and
generating a third multi-planar reformat (MPR) view from the CT data, the third MPR view being a view of CT data representative of a portion of the lung intersecting a third plane extending through the lung that includes the selected location in the selected lung segment, such that the third MPR view includes (i) a third planar portion of the selected lung segment containing the selected location and (ii) a portion of the lung, coplanar with the third planar portion, not corresponding to the selected lung segment; and
the display image simultaneously comprises the first MPR view, the second MPR view, and the third MPR view, wherein the display image:
visually distinguishes the first planar portion of the selected lung segment from the portion of the lung coplanar with the first planar portion and not corresponding to the selected lung segment in the first MPR view;
visually distinguishes the second planar portion of the selected lung segment from the portion of the lung coplanar with the second planar portion and not corresponding to the selected lung segment in the second MPR view; and
visually distinguishes the third planar portion of the selected lung segment from the portion of the lung coplanar with the third planar portion and not corresponding to the selected lung segment in the third MPR view.

3. The non-transitory computer readable medium of claim 1, wherein the MPR view comprises a view of CT data representative of a portion of the lung intersecting a first plane extending through the lung the method further comprises:
receiving, within the display image, a selection of a point of CT data within an airway in the display image;
identifying a first updated plane, the first updated plane being parallel to the first plane and containing the selected point of CT data;
generating a first updated MPR view of the CT data including CT data within the first updated plane and including the selected point;
identifying a second updated plane, the second updated plane being parallel to the second plane and containing the selected point of CT data;
generating a second updated MPR view of the CT data including CT data within the second updated plane and including the selected point;
identifying a third updated plane, the third updated plane being parallel to the third plane and containing the selected point of CT data;
generating a third updated MPR view of the CT data including CT data within the third updated plane and including the selected point; and
generating an updated display image comprising the first updated MPR view, the second updated MPR view, and the third updated MPR view.

4. The non-transitory computer readable medium of claim 1, wherein the method further comprises:
generating a topographic multi-planar reformat (tMPR) image from the set of CT data, the tMPR image comprising a two-dimensional representation of the selected lung segment; and
receiving a selection of a location within the tMPR image, the selected location corresponding to a selected location in the selected lung segment; and wherein
the generated MPR view from the CT data comprises CT data representative of a portion of the lung intersecting a plane extending through the lung that includes the selected location in the selected lung segment such that the MPR view includes a planar portion of the selected lung segment containing the selected location.

5. The non-transitory computer readable medium of claim 4, wherein the display image further comprises the generated tMPR image.

6. The non-transitory computer readable medium of claim 5, wherein the method further comprises:
receiving, within the tMPR image, a selection of a point of CT data within an airway in the tMPR image;
identifying a first updated plane, the first updated plane being parallel to the first plane and containing the selected point of CT data;
generating a first updated MPR view of the CT data including CT data within the first updated plane and including the selected point;
identifying a second updated plane, the second updated plane being parallel to the second plane and containing the selected point of CT data;
generating a second updated MPR view of the CT data including CT data within the second updated plane and including the selected point;
identifying a third updated plane, the third updated plane being parallel to the third plane and containing the selected point of CT data;
generating a third updated MPR view of the CT data including CT data within the third updated plane and including the selected point; and
generating an updated display image comprising the first updated MPR view, the second updated MPR view, and the third updated MPR view.

7. The non-transitory computer readable medium of claim 1, wherein visually distinguishing the planar portion of the selected lung segment from the portion of the lung coplanar with the planar portion and not corresponding to the selected lung segment comprises displaying the planar portion of the selected lung segment according to a first display criteria and displaying the portion of the lung coplanar with the planar portion and not corresponding to the selected lung segment according to a second display criteria.

8. The non-transitory computer readable medium of claim 7, wherein the first and second display criteria comprise one or more criteria from the group consisting of: color, transparency, focus/blur, obfuscation, and brightness.

9. The non-transitory computer readable medium of claim 8, wherein visually distinguishing the planar portion of the selected lung segment from the portion of the lung coplanar with the planar portion and not corresponding to the selected lung segment comprises obfuscating the portion of the lung coplanar with the planar portion and not corresponding to the selected lung segment.

10. The non-transitory computer readable medium of claim 9, wherein generating the display image comprises obfuscating CT data representing lung tissue within a predetermined margin from the chest wall.

11. The non-transitory computer readable medium of claim 10, wherein the predetermined margin is 2 cm such that CT data representing lung tissue within 2 cm of the chest wall is obfuscated from display in the display image.

12. The non-transitory computer readable medium of claim 8, wherein visually distinguishing the planar portion of the selected lung segment from the portion of the lung coplanar with the planar portion and not corresponding to the selected lung segment comprises highlighting the portion of the lung coplanar with the planar portion and not corresponding to the selected lung segment in a semi-transparent color.

13. The non-transitory computer readable medium of claim 7, wherein the selected lung segment comprises a sublobe.

14. The non-transitory computer readable medium of claim 1, wherein the method further comprises receiving an input characterizing the selected lung segment.

15. The non-transitory computer readable medium of claim 14, wherein the input characterizing the lung segment comprises an input related to whether a mucus plug is present within the selected lung segment.

16. The non-transitory computer readable medium of claim 14, wherein the input characterizing the lung segment comprises an input selecting one from a list of possible characterizations of the lung segment.

17. The non-transitory computer readable medium of claim 16, wherein the list of possible characterizations of the lung segment includes an indication of a presence and/or location of bronchiectasis, occlusion, and mucus plug.

18. The non-transitory computer readable medium of claim 14, wherein the selected lung segment comprises a first lung segment corresponding to a first subset of the set of CT data, and wherein the method further comprises determining whether all lung segments from a list of a plurality of lung segments have been characterized.

19. The non-transitory computer readable medium of claim 18, wherein the method further comprises, if not all lung segments from the list of the plurality of lung segments have been characterized:
receiving a selection of a second lung segment that corresponds to a second subset of the set of CT data;
generating an updated MPR view from the CT data, the updated MPR view being a view of CT data representative of a portion of the lung intersecting a plane extending through the lung in the selected second lung segment, such that the updated MPR view includes (i) an updated planar portion of the selected second lung segment and (ii) a portion of the lung, coplanar with the updated planar portion, not corresponding to the second selected lung segment;
generating an updated display image of the updated MPR view, the updated display image visually distinguishing the updated planar portion of the second selected lung segment from the portion of the lung coplanar with the updated planar portion and not corresponding to the second selected lung segment; and
receiving an input characterizing the second lung segment.

20. The non-transitory computer readable medium of claim 19, wherein the method further comprises, if not all lung segments from the list of the plurality of lung segments have been characterized:
generating a tMPR image from the second subset of CT data, the tMPR image comprising a two-dimensional representation of the second lung segment; and
receiving a selection of an updated location within the tMPR image, the selected updated location corresponding to a selected location in the selected second lung segment; and wherein
the updated MPR view from the CT data representative of a portion of the lung intersecting a plane extending through the lung includes the selected updated location such that the updated MPR view includes the updated planar portion of the selected second lung segment and contains the selected updated location.

21. The non-transitory computer readable medium of claim 1, wherein generating the display image comprises obfuscating CT data representing lung tissue within a predetermined margin from the chest wall.

22. A non-transitory computer readable medium comprising computer-readable instructions for causing one or more processors to perform a method for generating a visualization of volumetric data, the method comprising:
receiving a set of three-dimensional computerized tomographic (CT) data representative of a lung;
identifying a location of a candidate artifact present within an airway within the CT data;
generating a topographic multi-planar reformat (tMPR) image representative of a section of the CT data including the candidate artifact and a portion of the airway including the candidate artifact, the tMPR image comprising a 2-dimensional representation of the airway within the CT data;
generating a multi-planar reformat (MPR) view of the CT data, the MPR view of the CT data including CT data within a first plane and including at least a portion of the candidate artifact, the first plane being defined by a shape of the candidate artifact or a shape of the airway including the candidate artifact; and
generating a display image simultaneously comprising the tMPR image and the MPR view.

23. The non-transitory computer readable medium of claim 22, wherein the MPR view comprises a first MPR view and wherein the method further comprises:
generating a second MPR view of the CT data, the second MPR view of the CT data including CT data within a second plane and including at least a portion of the candidate artifact, the second plane being normal to the first plane;
generating a third MPR view of the CT data, the third MPR view of the CT data including CT data within a third plane and including at least a portion of the candidate artifact, the third plane being normal to the first plane and the second plane; and
wherein the display image simultaneously comprises the tMPR image and the first MPR view, the second MPR view, and the third MPR view.

24. The non-transitory computer readable medium of claim 23, wherein the method further comprises receiving a selection of a lung segment that corresponds to a subset of the set of CT data representative of the lung; and wherein
generating the display image comprises visually distinguishing the selected lung segment from CT data not corresponding to the selected lung segment in each the first MPR view, the second MPR view, and the third MPR view by displaying the selected lung segment according to a first display criteria and displaying CT data not corresponding to the selected lung segment according to a second display criteria.

25. The non-transitory computer readable medium of claim 24, wherein the first and second display criteria comprise one or more criteria from the group consisting of: color, transparency, focus/blur, obfuscation, and brightness.

26. The non-transitory computer readable medium of claim 25, wherein visually distinguishing the selected lung segment from the CT data not corresponding to the selected lung segment comprises obfuscating the CT data not corresponding to the selected lung segment.

27. The non-transitory computer readable medium of claim 25, wherein visually distinguishing the selected lung segment from the CT data not corresponding to the selected lung segment comprises highlighting the CT data not corresponding to the selected lung segment in a semi-transparent color.

28. The non-transitory computer readable medium of claim 24, wherein the selected lung segment comprises a sublobe.

29. The non-transitory computer readable medium of claim 23, wherein the method further comprises:
receiving, within the tMPR image, a selection of a point of CT data within an airway in the tMPR image;
identifying a first updated plane, the first updated plane being parallel to the first plane and containing the selected point of CT data;
generating a first updated MPR view of the CT data including CT data within the first updated plane and including the selected point;
identifying a second updated plane, the second updated plane being parallel to the second plane and containing the selected point of CT data;
generating a second updated MPR view of the CT data including CT data within the second updated plane and including the selected point;
identifying a third updated plane, the third updated plane being parallel to the third plane and containing the selected point of CT data;
generating a third updated MPR view of the CT data including CT data within the third updated plane and including the selected point; and
generating an updated display image comprising the tMPR image, the first updated MPR view, the second updated MPR view, and the third updated MPR view.

30. The non-transitory computer readable medium of claim 23, wherein the candidate artifact comprises a mucus plug candidate.

31. The non-transitory computer readable medium of claim 30, wherein the identifying the location of the candidate artifact comprises receiving location information representing a position of a mucus plug candidate in the CT data.

32. The non-transitory computer readable medium of claim 30, wherein the identifying the location of the candidate artifact comprises detecting one or more mucus plug candidates in the CT data using a neural network.

33. The non-transitory computer readable medium of claim 23, wherein the method further comprises receiving an input characterizing the airway.

34. The non-transitory computer readable medium of claim 33, wherein the method further comprises determining whether all candidate artifacts from a list of a plurality of identified candidate artifacts have been characterized, and if not all candidate artifacts from the list of the plurality of identified candidate artifacts have been characterized:
identifying a location of a second candidate artifact within a second airway within the CT data
generating an updated tMPR image representative of a second section of the CT data including the second candidate artifact and a second portion of the airway including the second candidate artifact, the updated tMPR image comprising a 2-dimensional representation of a second curved surface intersecting the CT data;
generating a first updated MPR view of the CT data, the first updated MPR view of the CT data including CT data within a first updated plane and including at least a portion of the second candidate artifact, the first updated plane being defined by the shape of the second candidate artifact or the shape of the second airway including the second candidate artifact;
generating a second updated MPR view of the CT data, the second updated MPR view of the CT data including CT data within a second updated plane and including at least a portion of the second candidate artifact, the second updated plane being normal to the first updated plane;
generating a third updated MPR view of the CT data, the third updated MPR view of the CT data including CT data within a third updated plane and including at least a portion of the second candidate artifact, the third updated plane being normal to the first updated plane and the second updated plane;
generating an updated display image simultaneously comprising the updated tMPR image, the first updated MPR view, the second updated MPR view, and the third updated MPR view; and
receiving an input characterizing the second airway.

35. The non-transitory computer readable medium of claim 23, wherein identifying the location of the candidate artifact comprises receiving a selection of a location in the CT data via a user interface.

36. The non-transitory computer readable medium of claim 22, wherein the MPR view comprises a first MPR view and the method further comprises:
generating a first translated MPR view of the CT data, the first translated MPR view including CT data within a first translated plane, the first translated plane being parallel to the first plane;
generating a second translated MPR view of the CT data, the second translated MPR view including CT data within a second translated plane, the second translated plane being parallel to the first plane and the first translated plane; and
toggling between the first MPR view, the first translated MPR view, and the second translated MPR view in the display image.

37. The non-transitory computer readable medium of claim 36, wherein the toggling between the first MPR view, the first translated MPR view, and the second translated MPR view in the display image is in response to a received input including instructions to translate the MPR view.
